(19) United States
(12) Patent Application Publication
(10) Pub. No.: US 2025/0244277 A1
(43) Pub. Date: Jul. 31, 2025

(54) METHOD OF EVALUATING ELECTRODE ACTIVITY

(71) Applicant: HONDA MOTOR CO., LTD., Tokyo (JP)

(72) Inventor: Toshiyuki OHASHI, Saitama (JP)

(21) Appl. No.: 18/991,738

(22) Filed: Dec. 23, 2024

(30) Foreign Application Priority Data
Jan. 31, 2024 (JP) 2024-012687

(51) Int. Cl. G01N 27/22; G01N 27/02; H01M 4/02

(52) U.S. Cl. CPC G01N 27/228; G01N 27/026; G01N 27/221; H01M 2004/021

(57) ABSTRACT

A method of evaluating electrode activity according to one embodiment of the present invention includes making evaluation of activity of an electrode that includes an active material, the activity of the electrode being evaluated based on the specific capacitance and specific surface area of an active material film formed solely from the active material, and the specific capacitance of the electrode.

This application is based on and claims the benefit of priority from Japanese Patent Application No. 2024-012687, filed on 31 Jan. 2024, the content of which is incorporated herein by reference.

### BACKGROUND OF THE INVENTION

### Field of the Invention

The present invention pertains to a method of evaluating electrode activity.

### Related Art

In recent years, research and development pertaining to secondary batteries that contribute to improving energy efficiency has been carried out in order to be able to ensure access to sustainable and advanced energy that is affordable and can be trusted by many people. Secondary batteries are greatly anticipated as an energy source that will replace conventional fossil fuels, and development of vehicle-mounted batteries in particular has been increasingly heating up in recent years. Various characteristics are required for vehicle-mounted batteries, but of these reduced resistance of various members inside a battery is important and directly relates to performance or cost, and thus development of various materials is proceeding. In parallel with such material development, methods of evaluating battery performance when a battery is configured by various members are also being studied. For example, methods that use an electrochemical method to evaluate the active surface area of an electrode used in a fuel cell are being studied (Patent Documents 1 and 2). A method of monitoring electrochemical capacitance using an electrochemical impedance method has been considered as an index for particle cracking of nickel-rich lithium nickel manganese cobalt oxide (NMC) used in a positive electrode for a lithium-ion secondary battery (Non-Patent Document 1).
Patent Document 1: Japanese Unexamined Patent Application, Publication No. 2011-228131
Patent Document 2: Japanese Unexamined Patent Application, Publication No. 2004-220786
Non-Patent Document 1: Stefan Oswald, Daniel Pritzl, Morten Wetjen, Hubert A. Gasteiger, Novel Method for Monitoring the Electrochemical Capacitance by In Situ Impedance Spectroscopy as Indicator for Particle Cracking of Nickel-Rich NCMs: Part I. Theory and Validation, Journal of The Electrochemical Society, 167, 100511 (2020)

### SUMMARY OF THE INVENTION

Improving an electrical characteristic such as an output characteristic is one problem for secondary batteries. Developing a method that can evaluate electrode activity is desired in order to improve electrical characteristics of a secondary battery. However, an electrode used in a lithium-ion secondary battery is a mixture that includes an electrode active material, a binder, and an electrically conductive aid. Furthermore, in the case of a nonaqeuous solvent battery, an electrode is incorporated in the battery in a state of being impregnated with a liquid electrolyte. In addition, in the case of an all-solid-state battery, a solid electrolyte may be added to an electrode. In this manner, a contact interface between an electrolyte and active material within an electrode is complex and spatially wide across the entire surface of the electrode. Accordingly, it is not easy to extract the interface between an electrolyte and active material within an electrode, and evaluating electrode activity is difficult.

The present invention is made in light of the situation described above, and an object of the present invention is to provide a method that can accurately evaluate activity of an electrode that includes an active material that occludes and discharges a charge transfer medium, such as an active material for a lithium-ion battery. Consequently, another object of the present invention is to contribute to improving energy efficiency.

The inventors found that it was possible to measure the specific capacitance and specific surface area of an active material film formed solely from an active material used in an electrode to be evaluated, and use this data regarding the active material film and the specific capacitance of the electrode to be evaluated to thereby accurately evaluate activity of the electrode to be evaluated, and thus completed the present invention. Accordingly, the present invention provides the following method.

A first aspect of the present invention is directed to a method of evaluating activity of an electrode that includes an active material, the method including making evaluation of activity of the electrode, based on a specific capacitance and a specific surface area of an active material film formed solely from the active material, and a specific capacitance of the electrode.

By virtue of the method of evaluating electrode activity according to the first aspect, activity of an electrode is evaluated based on the specific capacitance of the electrode, as well as the specific surface area and specific capacitance of an active material film, which is formed solely from an active material, and thus it is possible to accurately evaluate activity of an electrode to be evaluated.

According to a second aspect of the present invention, in the method of evaluating electrode activity according to the first aspect, the evaluation of the activity of the electrode being performed in accordance with an electrode active specific surface area (m²/g) calculated using the following formula (I),

$$Electrode active specific surface area ( {m}^{2} / g ) = a \times x + b \qquad ( I )$$

in formula (I), a is a gradient of a calibration curve for the specific capacitance and specific surface area of the active material film, b is a specific surface area of the electrode when a surface roughness Ra of the electrode was set to zero, and x is the specific capacitance of the electrode.

By virtue of the method of evaluating electrode activity according to the second aspect, activity of an electrode is evaluated using the electrode active specific surface area calculated using formula (I), whereby it is possible to accurately evaluate activity of an electrode to be evaluated.

According to a third aspect of the present invention, in the method of evaluating electrode activity according to the first or second aspect, the specific capacitance of the active material film being calculated by using an electrochemical impedance method to measure a capacitance of the active material film, and dividing the obtained capacitance by a mass of the active material film.

By virtue of the method of evaluating electrode activity according to third aspect, it is possible to accurately measure the specific capacitance of an active material film, and thus it is possible to more accurately evaluate activity of an electrode to be evaluated.

According to a fourth aspect of the present invention, in the method of evaluating electrode activity according to any one of the first through third aspects, the specific surface area of the active material film being calculated by using surface probe microscopy to measure the surface area of the active material film, and dividing the obtained surface area by a mass of the active material film.

By virtue of the method of evaluating electrode activity according to the forth aspect, it is possible to accurately measure the specific surface area of an active material film, and thus it is possible to more accurately evaluate activity of an electrode to be evaluated.

According to a fifth aspect of the present invention, in the method of evaluating electrode activity according to any one of the first to fourth aspects, the electrode being for a battery that uses a liquid electrolyte.

By virtue of the method of evaluating electrode activity according to the fifth aspect, it is possible to evaluate activity of an electrode for a battery that uses a liquid electrolyte.

According to a sixth aspect of the present invention, in the method of evaluating electrode activity according to the fifth aspect, the specific capacitance of the electrode being calculated by using an electrochemical impedance method to measure a capacitance of the electrode in a state where the liquid electrolyte used in the battery that uses the electrode is caused to be in contact with the electrode, and the obtained capacitance being divided by a mass of the active material in the electrode.

By virtue of the method of evaluating electrode activity according to the sixth aspect, the specific capacitance of the electrode is measured in a state close to that of the battery that uses the liquid electrolyte, and thus it is possible to more accurately evaluate activity of an electrode in a battery that uses a liquid electrolyte.

According to a seventh aspect of the present invention, in the method of evaluating electrode activity according to any one of the first to fourth aspects, the electrode being for a battery that uses a solid electrolyte.

By virtue of the method of evaluating electrode activity according to the seventh aspect, it is possible to evaluate activity of an electrode for an all-solid-state battery that uses a solid electrolyte.

According to an eighth aspect of the present invention, in the method of evaluating electrode activity according to the seventh aspect, the specific capacitance of the electrode being calculated by using an electrochemical impedance method to measure a capacitance of the electrode in a state where the solid electrolyte used in the battery that uses the electrode is caused to be in contact with the electrode, and the obtained capacitance being divided by a mass of the active material in the electrode.

By virtue of the method of evaluating electrode activity according to the eighth aspect, the specific capacitance of the electrode is measured in a state close to that of a solid-state secondary battery that uses the solid electrolyte, and thus it is possible to more accurately evaluate activity of an electrode in a solid-state secondary battery.

By virtue of the present invention, it becomes possible to provide a method that can accurately evaluate activity of an electrode that includes an active material that occludes and discharges a charge transfer medium, such as an active material for a lithium-ion battery.

### BRIEF DESCRIPTION OF THE DRAWINGS

FIG. 1 is a graph illustrating a calibration curve for the specific capacitance and specific surface area of a positive electrode active material thin film prepared in example 1.

### DETAILED DESCRIPTION OF THE INVENTION

Description is given below regarding an embodiment pertaining to a method of evaluating electrode activity according to the present invention. The method of evaluating electrode activity according to the present embodiment evaluates activity of an electrode that includes an active material. An electrode to be evaluated, for example, may be for a battery that uses a solid electrolyte, or may be for a battery that uses a liquid electrolyte. For example, a battery may be a lithium battery that uses lithium as a charge transfer medium. An active material may be a positive electrode active material, or may be a negative electrode active material.

The method of evaluating electrode activity according to the present embodiment evaluates activity of an electrode to be evaluated, based on the specific capacitance of the electrode to be evaluated, as well as the specific surface area and specific capacitance of an active material film, which is formed solely from an active material that is used in the electrode to be evaluated.

Evaluation of the activity of an electrode to be evaluated can be performed in accordance with an electrode active specific surface area (m²/g) calculated using the following formula (I), for example.
(I) Electrode active specific surface area (m²/g)=a×x+b In formula (I), a is the gradient of a calibration curve for the specific capacitance and specific surface area of an active material film, b is a specific surface area resulting from dividing the surface area of the electrode when the surface roughness Ra of the electrode is set to zero by the mass of the active material included in the electrode, and x is the specific capacitance of the electrode.

At the interface between an electrode and an electrolyte within a battery, two materials having different moduli of elasticity are in contact. It is possible to use surface analysis of the surface roughness of the electrode to obtain the surface area of the contact interface between the electrode and the electrolyte. However, in a typical battery, the electrode and the electrolyte are in contact in a complex manner and have spatial wideness. Therefore, it is difficult to perform surface analysis of the surface roughness of the electrode in a non-destructive manner. Accordingly, in the present embodiment, the above formula (I) is used to associate the specific capacitance of an electrode to be evaluated with the contact area at the interface between the electrode to be evaluated and an electrolyte. In other words, the specific capacitance of the electrode to be evaluated is assigned to x in (I) above, whereby the electrode active specific surface area, which serves as an index for the contact area at the interface between the electrolyte and the active material in the electrode to be evaluated, is calculated. In addition, a in the above formula (I) is a gradient obtained from a calibration curve of the specific surface area and specific capacitance of an active material film that is formed from the active material. Therefore, there ceases to be a need to separately consider capacitance values for each material such as the active material, binder, and electrically conductive aid included in an ordinary electrode. Accordingly, it is possible to use the above formula (I) to quantitatively ascertain the electrode active specific surface area of an electrode to be evaluated, without considering a specific capacitance other than that of the active material. In other words, the electrode active specific surface area, which serves as an index for the contact area at the interface between the electrolyte and the active material in the electrode to be evaluated, can be obtained from the specific capacitance value of the electrode to be evaluated.

The electrode active specific surface area can be obtained using a method that includes the following steps, for example.
(1) Step for preparing active material film
(2) Measurement step for measuring the specific surface area and specific capacitance of the active material film
(3) Calculation step for calculating a gradient of calibration curve for the specific capacitance and specific surface area of the active material film
(4) Step for measuring the specific capacitance of an electrode to be evaluated
(5) Step for calculating the specific surface area of the electrode to be evaluated when the surface roughness Ra of the electrode to be evaluated is set to zero
(6) Calculation step for calculating the electrode active specific surface area

In step (1), an active material film that is formed from an active material alone is prepared. The active material film can be formed on an electrically conductive substrate. The active material film may be a thin film having a thickness that is within the range of greater than or equal to 20 nm and less than or equal to 1 μm, for example.

A method of preparing the active material film may be a dry process, or may be a wet process. It is possible to use pulsed-laser vapor deposition method (PLD method) or RF sputtering as the dry process. As the wet process, it is possible to use a method for generating an active material in which an active material precursor solution is coated to a substrate, and an obtained coating film is baked. In addition, in a case where an active material used in an electrode to be evaluated has a coating layer on the surface thereof, a coating layer may be formed on the surface of the active material film.

In step (1), three or more types of active material films that have different surface roughnesses Ra may be prepared in order to prepare a calibration curve for the specific capacitance and specific surface area of the active material film in step (3), which is described below.

In step (2), the specific surface area and the specific capacitance of the active material films obtained in step (1) are measured.

The specific surface area of an active material film can be obtained by, for example, measuring the surface area of the active material film and the mass of the active material film, and dividing the obtained surface area of the active material film by the mass of the active material film. It is possible to use surface probe microscopy, for example, to measure the surface area of an active material film. Additional explanation will be given here regarding the specific surface area. The specific surface area of an active material film described above is used above in a case where the modulus of elasticity of the active material film is greater than or equal to the modulus of elasticity of a solid electrolyte. This is because, in this case, the contact interface between the positive electrode and the solid electrolyte within the battery depends on the surface roughness of the positive electrode. In contrast, the specific surface area of the solid electrolyte is used above in a case where the modulus of elasticity of the active material film is less than the modulus of elasticity of the solid electrolyte. This is because, in this case, the contact interface between the positive electrode and the solid electrolyte within the battery depends on the surface roughness of the solid electrolyte. The specific surface area of the active material film can be obtained by measuring the surface area of the active material film and dividing the surface area by the mass of the active material film. The specific surface area of the solid electrolyte film can be obtained by, for example, measuring the surface area of the solid electrolyte film, and dividing the obtained surface area of the solid electrolyte film by the mass of the active material film.

The specific capacitance of the active material film can be measured as follows in a case where the active material is a positive electrode active material, for example. Firstly, an active material film cell is prepared, the active material film cell employing the active material film as a positive electrode, employing lithium or a lithium alloy as a negative electrode, and disposing an electrolyte between the positive electrode and the negative electrode. Next, the capacitance of the positive electrode is measured. The specific capacitance is then calculated by dividing the obtained capacitance by the mass of the active material film. The electrolyte in the active material film cell can be the same as the electrolyte in the battery in which the electrode to be evaluated is used. It is possible to use an electrochemical impedance method as a method of measuring the capacitance.

In step (3), a calibration curve is created using the specific surface area and the specific capacitance of the active material film obtained in step (2). For the calibration curve, the specific capacitance is set to the horizontal axis, and the specific surface area is set to the vertical axis. An approximate formula (linear regression formula) for the obtained calibration curve is determined, and the gradient of the obtained approximate formula is obtained.

In step (4), the specific capacitance of the electrode to be evaluated is measured. In a case where the electrode to be evaluated is a positive electrode, for example, the specific capacitance of the electrode to be evaluated can be measured as follows. Firstly, an electrode cell to be evaluated is prepared, the electrode cell to be evaluated employing the electrode to be evaluated as a positive electrode, employing lithium or a lithium alloy as a negative electrode, and disposing an electrolyte between the positive electrode and the negative electrode. Next, the capacitance of the electrode to be evaluated is measured. The specific capacitance is then calculated by dividing the obtained capacitance by the mass of the active material film in the electrode to be evaluated. The electrolyte in the electrode cell to be evaluated can be the same as the electrolyte in the active material film cell. It is possible to use an electrochemical impedance method as a method of measuring the capacitance.

In step (5), the specific surface area of the electrode to be evaluated when the surface roughness Ra of the electrode to be evaluated is set to zero is calculated. Specifically, the specific surface area is calculated by dividing the geometric surface area of the electrode to be evaluated by the active material content of the electrode to be evaluated.

In step (6), the electrode active specific surface area is obtained by assigning the specific capacitance of the electrode to be evaluated obtained in step (4) to x in the following formula (I), for which the gradient of the calibration curve of the specific capacitance and specific surface area of the active material film obtained in step (3) is set to a, and the specific surface area of the electrode to be evaluated obtained in step (5) is set to b.

Electrode active specific surface area(m²/g)=a×x+b (I)

The electrode active specific surface area serves as an index for the contact area at the interface between the active material and the electrolyte in the electrode to be evaluated. Accordingly, a higher electrode active specific surface area means that the contact area at the interface between the active material and the electrolyte is larger, and an electrode reaction by the active material is more likely to occur. Accordingly, an electrode having a large electrode active specific surface area improves an output characteristic.

By virtue of the method of evaluating electrode activity that is according to the present embodiment and is configured as above, activity of an electrode is evaluated based on the specific capacitance of an electrode to be measured, as well as the specific surface area and specific capacitance of an active material film, which is formed solely from an active material, and thus it is possible to accurately evaluate activity of an electrode to be evaluated. In addition, by virtue of the method of evaluating electrode activity according to the present embodiment, activity of an electrode is evaluated using the electrode active specific surface area calculated using formula (I), whereby it is possible to accurately evaluate activity of an electrode to be evaluated. By virtue of the method of evaluating electrode activity according to the present embodiment, it is possible to evaluate activity of either an electrode for a solid electrolyte battery, or an electrode for a battery that uses a liquid electrolyte.

By virtue of the method of evaluating electrode activity according to the present embodiment, it becomes possible to use the electrochemical impedance method to measure the capacitance of an active material film and perform a calculation by dividing the obtained capacitance by the mass of the active material film to thereby accurately measure the specific capacitance of the active material film, and thus it is possible to more accurately evaluate activity of the electrode to be evaluated. In addition, by virtue of the method of evaluating electrode activity according to the present embodiment, it becomes possible to calculate the specific surface area of an active material film by using surface probe microscopy to measure the surface area of the active material film and dividing the obtained surface area by the mass of the active material film and thereby accurately measure the specific surface area of the active material film, and thus it is possible to more accurately evaluate activity of an electrode to be evaluated.

In the method of evaluating electrode activity according to the present embodiment, in a case where an electrode to be evaluated is for a battery that uses a liquid electrolyte, the specific capacitance of the electrode to be evaluated is calculated by using an electrochemical impedance method to measure the capacitance of the electrode to be evaluated in a state where the liquid electrolyte is caused to be in contact with the electrode, and dividing the obtained capacitance by the mass of the active material in the electrode to be evaluated, whereby it is possible to more accurately evaluate activity of the electrode to be evaluated in the battery. In the method of evaluating electrode activity according to the present embodiment, in a case where an electrode to be evaluated is for a solid-state secondary battery that uses a solid electrolyte, the specific capacitance of the electrode to be evaluated is calculated by using an electrochemical impedance method to measure the capacitance of the electrode to be evaluated in a state where the solid electrolyte is caused to be in contact with the electrode, and dividing the obtained capacitance by the mass of the active material in the electrode to be evaluated, whereby it is possible to more accurately evaluate activity of the electrode to be evaluated in the battery.

### EXAMPLES

The present invention is described below based on an example. However, the present invention is not limited to this example.

### Example 1

Activity of an electrode to be evaluated that includes NMC (composition: LiNi_{1/3}Co_{1/3}Mn_{1/3}O₂) coated by LiNbO₃ as a positive electrode active material was evaluated in the following manner.

### <Preparation of Positive Electrode Active Material Thin Film>

A result of sputter deposition a Pt current collection layer on the entire surface of an Si single-crystal substrate (10 mm longitudinal×10 mm lateral) is prepared as a current collection substrate. Firstly, a LiNi_{1/3}Co_{1/3}Mn_{1/3}O₂(NMC) thin film (9 mm longitudinal×9 mm lateral) is prepared by the pulsed-laser vapor deposition method (PLD method) using a sintered body of NMC as a target, on one surface of the abovementioned current collection substrate. Film formation conditions for the NMC thin film are, under the chamber oxygen gas pressure indicated in Table 1, setting the temperature to 650° C., setting an amount of holding time to 50 minutes, employing a Nd-YAG laser (wavelength of 266 nm), setting output to 200 mW, and setting the oscillation frequency to 10 Hz.

Next, a LiNbO₃ coat layer (9 mm longitudinal×9 mm lateral×several nm thick) is formed on the NMC film by PLD using an LiNbO₃ sintered body as a target. Film formation conditions for the LiNbO₃ coat layer are the same as the film formation conditions for the NMC thin film, apart from setting the temperature to 400° C. and setting the amount of holding time to 15 minutes. In this manner, positive electrode active material thin films having the sample numbers 1 to 3 indicated in Table 1 were obtained.

TABLE 1

| SAMPLE NUMBER OF | |
|---|---|
| POSITIVE ELECTRODE | CHAMBER OXYGEN |
| ACTIVE MATERIAL | GAS PRESSURE |
| THIN FILM | (Pa) |
| 1 | 10.5 |
| 2 | 9.99 |
| 3 | 10.1 |

### <Measurement of Specific Surface Area and Specific Capacitance of Positive Electrode Active Material Thin Film>

The specific surface area and specific capacitance were measured, as described below, for the positive electrode active material thin films having sample numbers 1 to 3. The results are indicated in Table 2.

### (Method of Measuring Specific Surface Area)

A surface roughness Ra value for the positive electrode active material thin film is obtained in a tapping mode for a surface probe microscope. The surface area of the positive electrode active material thin film is calculated from the obtained surface roughness Ra value. In addition, a precision balance is used to measure the weight of the current collection substrate on which the positive electrode active material thin film has been formed, and the weight of the current collection substrate, which is measured in advance, is subtracted to thereby calculate the weight of the positive electrode active material thin film. The specific surface area is obtained by dividing the surface area by the weight of the positive electrode active material film.

### (Method of Measuring Specific Capacitance)

The positive electrode active material thin-film cell is prepared as follows. The current collection substrate on which the positive electrode active material thin film has been formed is disposed. Next, a cylindrical container having an opening that is 16 mm in diameter is mounted to a pressurizing shaft. Subsequently, 250 mg of an argyrodite-based sulfide (composition: Li_{5.5}PS_{4.5}Cl_{1.5}) powder was supplied onto the positive electrode active material thin film. A different pressurizing shaft is inserted from the other end of the cylindrical container, a molding pressure indicated in Table 2 is applied to the argyrodite-based sulfide powder by the pressurizing shaft, and a sulfide-based solid electrolyte layer formed from the argyrodite-based sulfide powder is formed on the positive electrode active material thin film. Next, the pressurizing shaft that was inserted later is pulled out, a lithium-indium foil (a Li—In foil) is disposed on the pressurizing shaft that was pulled out, then the pressurizing shaft on which the Li—In foil was disposed is reinserted, and a molding pressure of 115 MPa is applied using the pressurizing shaft. In this manner, a positive electrode active material thin-film cell is prepared, the current collection substrate on which the positive electrode active material thin film is formed being employed as a positive electrode, the Li—In foil being employed as a negative electrode, and the sulfide-based solid electrolyte layer being disposed between the positive electrode and the negative electrode.

The capacitance of the positive electrode active material thin film in the obtained positive electrode active material thin-film cell is measured as follows. Firstly, the positive electrode active material thin-film cell is connected to a charge/discharge apparatus. Three-cycle charging and discharging is performed with respect to the thin-film cell, at a 1 μA constant current (CC) and in a voltage range of 2.08 V to 3.68 V. Capacitance is measured during discharge of the third cycle to thereby calculate a C rate. Next, the positive electrode active material thin-film cell is connected to a potentio-galvanostat apparatus that has a function for measuring electrochemical impedance (EIS). Firstly, discharge is caused at a 0.1 C constant current discharge (CC) until 2.08 V. When 2.08 V is reached, constant current/constant voltage discharge (CC-CV) is performed until the residual current becomes less than or equal to 0.01 C. Next, the positive electrode active material thin-film cell is left alone for three hours, and then the capacitance of the positive electrode active material thin film is measuring using the EIS method. EIS measurement conditions are frequency range: 1 MHz to 10 mHz, and alternating-current voltage amplitude: 10 mV. EIS measurement is performed at a temperature of 25° C. after inserting the positive electrode active material thin-film cell into a thermostatic tank.

A capacitance value is obtained, based on a Z″ value that is near 180 mHz and is obtained by EIS measurement, and specific capacitance is obtained by dividing the capacitance value by the weight of the previously-described positive electrode active material thin film.

TABLE 2

| SAMPLE NUMBER OF | SPECIFIC | | SPECIFIC |
|---|---|---|---|
| POSITIVE ELECTRODE | SURFACE | MOLDING | CAPACI- |
| ACTIVE MATERIAL | AREA | PRESSURE | TANCE |
| THIN FILM | (m²/g) | (MPa) | (F/g) |
| 1 | 9.62 | 290 | 6.83 |
| 2 | 6.33 | 150 | 1.40 |
| 3 | 7.22 | 150 | 2.40 |

### <Calculation of Gradient of Calibration Curve for Specific Capacitance and Specific Surface Area of Positive Electrode Active Material Thin Film>

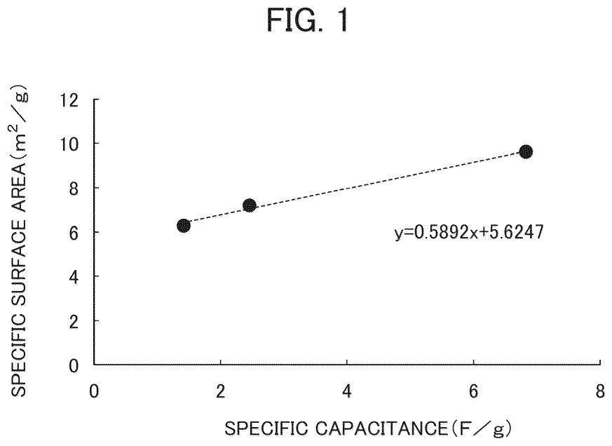

A calibration curve that employs the specific capacitance indicated in Table 2 as the horizontal axis, and the specific surface area as the vertical axis is created. The obtained calibration curve is illustrated in FIG. 1. An approximate formula (linear regression formula) for the obtained calibration curve was y=0.5892x+5.6247. From this approximate formula, it is ascertained that the specific capacitance and the specific surface area of the positive electrode active material thin film are correlated in a relationship that has a gradient of 0.5892.

### <Preparation of Electrode to be Evaluated>

A composite layer is formed on an aluminum current collector foil, the composite layer including positive electrode active material powder (NMC coated by LiNbO₃), argyrodite-based sulfide solid electrolyte powder, an electrically conductive aid (acetylene black), and a binder (styrene-butadiene rubber) at a mass ratio in 75:21:3:1 proportions. The positive electrode active material powder content in the composite layer was 16 mg. The obtained aluminum current collector foil that has the composite layer was punched out into a circular shape having a diameter of 10 mm to thereby obtain an electrode to be evaluated.

### <Measurement of Specific Capacitance of Electrode to be Evaluated>

The electrode cell to be evaluated was prepared as follows. An electrode to be evaluated was inserted and disposed within a cylindrical container having a circular opening that is 10 mm in diameter. Next, 100 mg of argyrodite-based sulfide (composition: Li_{5.5}PS_{4.5}Cl_{1.5}) powder was supplied onto the composite layer of the electrode to be evaluated. A pressurizing shaft was inserted into each of the both openings of the cylindrical container, a 100 MPa molding pressure is applied to the argyrodite-based sulfide powder using the pressurizing shafts, and a sulfide-based solid electrolyte layer formed from the argyrodite-based sulfide powder is formed on the composite layer of the electrode to be evaluated. Next, the sulfide-based solid electrolyte layer side pressurizing shaft was pulled out, a lithium-indium foil (a Li—In foil) was disposed on the sulfide-based solid electrolyte layer, then the pressurizing shaft was reinserted onto the Li—In foil, and a 150 MPa molding pressure was applied using the pressurizing shaft. In this manner, an electrode cell to be evaluated was prepared, the electrode to be evaluated being employed as a positive electrode, the Li—In foil being employed as a negative electrode, and a sulfide-based solid electrolyte layer being disposed between the positive electrode and the negative electrode.

The capacitance of the composite layer in the obtained electrode cell to be evaluated was measured similarly to the capacitance of the positive electrode active material thin film described above. The specific capacitance of the positive electrode active material powder in the electrode cell to be evaluated was calculated by dividing the obtained capacitance of the composite layer by the positive electrode active material powder content in the composite layer. The obtained specific capacitance was 0.28 F/g.

<Calculation of Specific Surface Area of Positive Electrode Active Material Powder when Surface Roughness Ra of the Electrode to be Evaluated is Zero>

A value resulting from dividing the geometric surface area of the electrode to be evaluated by the positive electrode active material powder content in the composite layer was calculated as the specific surface area of the positive electrode active material powder when the surface roughness Ra is zero. The obtained specific surface area of the positive electrode active material powder when the surface roughness Ra was zero was 0.005 m²/g.

### <Calculation of Active Specific Surface Area of Positive Electrode Active Material Powder in Electrode to be Evaluated>

The following formula (II) was obtained as a relational expression between the specific capacitance and the active specific surface area of the positive electrode active material powder in the electrode to be evaluated, from the specific surface area of the positive electrode active material powder when the surface roughness Ra was zero (0.005 m²/g), and the gradient for the specific capacitance and the specific surface area of the positive electrode active material thin film (0.5892).

y=0.5892x+0.005 (II)

The electrode active specific surface area y of the positive electrode active material powder in the electrode to be evaluated was calculated by assigning the specific capacitance of the positive electrode active material powder in the electrode to be evaluated (0.28 F/g) to x in formula (II) above. As a result, the electrode active specific surface area y was 0.17 m²/g.

What is claimed is:

1. A method of evaluating activity of an electrode that includes an active material, the method comprising:
making evaluation of activity of the electrode, based on a specific capacitance and a specific surface area of an active material film formed solely from the active material, and
a specific capacitance of the electrode.

2. The method of evaluating electrode activity according to claim 1,
wherein the evaluation of the activity of the electrode is performed in accordance with an electrode active specific surface area (m²/g) calculated using the following formula (I),
Electrode active specific surface area(m²/g)=a×x+b (I)
in formula (I), a is a gradient of a calibration curve for the specific capacitance and specific surface area of the active material film, b is a specific surface area of the electrode when a surface roughness Ra of the electrode was set to zero, and x is the specific capacitance of the electrode.

3. The method of evaluating electrode activity according to claim 1,
wherein the specific capacitance of the active material film is calculated by using an electrochemical impedance method to measure a capacitance of the active material film, and
dividing the obtained capacitance by a mass of the active material film.

4. The method of evaluating electrode activity according to claim 1,
wherein the specific surface area of the active material film is calculated by using surface probe microscopy to measure the surface area of the active material film, and
dividing the obtained surface area by a mass of the active material film.

5. The method of evaluating electrode activity according to claim 1,
wherein the electrode is for a battery that uses a liquid electrolyte.

6. The method of evaluating electrode activity according to claim 5,
wherein the specific capacitance of the electrode is calculated by using an electrochemical impedance method to measure a capacitance of the electrode in a state where the liquid electrolyte used in the battery that uses the electrode is caused to be in contact with the electrode, and
the obtained capacitance is divided by a mass of the active material in the electrode.

7. The method of evaluating electrode activity according to claim 1,
wherein the electrode is for a battery that uses a solid electrolyte.

8. The method of evaluating electrode activity according to claim 7,
wherein the specific capacitance of the electrode is calculated by using an electrochemical impedance method to measure a capacitance of the electrode in a state where the solid electrolyte used in the battery that uses the electrode is caused to be in contact with the electrode, and
the obtained capacitance is divided by a mass of the active material in the electrode.
